(19) United States
(12) Patent Application Publication
(10) Pub. No.: US 2010/0120729 A1
(43) Pub. Date: May 13, 2010

(54) PHEROMONE-CONTAINING COSMETIC AGENTS

(76) Inventor: Rolf Bayersdörfer, Hamburg (DE)

Correspondence Address: Henkel Corporation, 10 Finderne Avenue, Bridgewater, NJ, 08807, US

(21) Appl. No.: 12/617,865

(22) Filed: Nov. 13, 2009

Related U.S. Application Data
(63) Continuation of application No. PCT/EP2008/051590, filed on Feb. 11, 2008.

(30) Foreign Application Priority Data
May 14, 2007 (DE) 10 2007 022 916.1

(51) Int. Cl. A61K 31/5685; A61K 31/56; A61K 31/568; A61Q 5/00; A61Q 19/00; A61Q 90/00

(52) U.S. Cl. 514/170; 514/169; 514/177; 514/182

(57) ABSTRACT

Cosmetic substances having 1x×10⁻⁷ to 1×10⁻² percent by weight of at least one steroid chosen from among certain 16-androstene and estrene steroids, and 0.1 to 30.0 percent by weight of at least one film-forming and/or solidifying polymers. Also disclosed is the use of those cosmetic substances for treating keratin fibers, particularly for temporarily deforming such fibers.

### CROSS REFERENCE TO RELATED APPLICATIONS

The present application is a continuation of International Application No. PCT/EP2008/051590, filed 11 Feb. 2008, which claims the benefit of German Patent Application No. 10 2007 022 916.1, filed 14 May 2007.

The present invention relates to cosmetic agents containing specific pheromones and at least one film-forming and/or setting polymer, and to a method of using these agents for treating keratin fibers, in particular for temporary deformation of these fibers.

Keratin fibers refer to all kinds of animal hair, for example, wool, horsehair, angora hair, furs, feathers, as well as products or textiles manufactured therefrom. Preferably, the keratin fibers comprise human hair.

Modern life is unimaginable without cosmetic agents. They function in cleaning, caring for and decorating the body, in particular the skin and hair, and thus, ultimately in promoting a user's well-being.

Depending on its purpose, a cosmetic agent can contain a variety of ingredients, including a range of active substances having an intrinsic odor which the user considers unpleasant. In order to overcome such odors or to increase the attractiveness of a cosmetic agent by imparting a pleasant odor experience, individual fragrances or fragrance mixtures or “perfume compositions” have long been added to cosmetic agents. However, it is possible for an individual fragrance or a component of the perfume composition to have an odor in its pure form or at elevated concentration which is considered unpleasant. Still, if the respective components are used in an optimized concentration and/or mixture, the desired pleasant odor experience is obtained.

Many highly diverse chemical compounds may be considered as fragrances, including naturally occurring compounds extracted from plants. A major role is, however, also played by nature-identical and artificial fragrances. Fragrances which, in addition to their fragrancing characteristic, have further desired characteristics, for instance conditioning or antioxidant actions, are of particular interest.

Use of pheromones in perfume compositions and cosmetic agents has previously been proposed in this connection. The term pheromone refers to signaling substances, active even at extremely low concentrations, which serve in chemical communication between members of the same species. Pheromones are involved in the communications systems of virtually all living organisms, from unicellular organisms up to mammals and play a major role as sexual attractants.

U.S. Pat. Nos. 5,272,134 and 5,278,141 describe the use of specific human pheromones of the 16-androstene steroid and estrene steroid type in perfume compositions. Possible fields of application mentioned include not only personal hygiene products, but also household and industrial products.

Use of these human pheromones in cosmetic agents is of interest because they not only impart a characteristic odor, but they are also capable, in their function as a signaling substance, of binding to the corresponding receptors in the human body. A corresponding agent or the user thereof therefore purposefully imparts to other humans, in particular humans of the opposite sex, natural signals which can have an influence on the evaluation of the attractiveness of the agent or of the user. However, a disadvantage of using these pheromones is that their intrinsic odor is generally considered to be somewhat unpleasant, as may be inferred from WO 2004/009051 A2, which relates to possible ways of specifically suppressing the natural formation of these pheromones by using suitable perfume components.

The present invention provides cosmetic agents which permit the use of corresponding human pheromones without releasing an unpleasant odor. It should furthermore be ensured that the desired action of the pheromone is retained over an extended period after application and that moreover no unpleasant odor develops within this period.

It has now been found that this may be achieved by cosmetic agents which, in addition to the pheromone, contain at least one film-forming and/or setting polymer.

The present invention accordingly firstly provides cosmetic agents containing, in a cosmetically acceptable carrier—

a) 1×10⁻⁷ to 1×10⁻² wt. % of at least one steroid chosen from
16-androstene steroids of the formula (I)

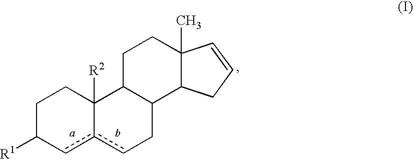

wherein R¹ denotes an oxo group, a hydroxyl group, a C₁-C₆ alkoxy, an acyloxy, or a benzoyloxy group; R² denotes hydrogen, a hydroxyl group, a C₁-C₆ alkyl, hydroxy-C₁-C₆-alkyl, C₁-C₆-alkoxy-C₁-C₆-alkyl, C₁-C₆ alkoxy, acyl, acyloxy, acyl-C₁-C₆-alkyl or acyloxy-C₁-C₆-alkyl group; and the dashed lines a and b denote alternative positions of a double bond, and

estrene steroids of the formula (II)

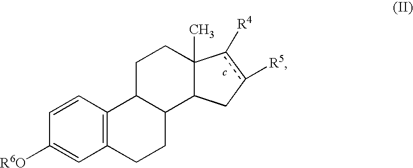

wherein R⁴ denotes an oxo group, hydrogen, a hydroxyl group, a C₁-C₆ alkyl group, sulfate, a 3-cyclopentylpropionate group or acetate; R⁵ denotes hydrogen or a hydroxyl group; R⁶ denotes hydrogen, a C₁-C₆ alkyl group, a benzoyl, 3-cyclopentylpropionate, acetyl, C₁-C₆ acyl or a sulfate group; and the dashed line c denotes an optional double bond, and
   b) 0.1 to 30 wt. % of at least one film-forming and/or setting polymer.

Use of the film-forming and/or setting polymer enables the pheromone to be continuously released only at very low concentrations. Further, this ensures that an unpleasant odor is not formed while obtaining a long-lasting effect. In addition, the pheromone is not prematurely released by normal environmental influences, such as wind and moisture, or by body heat and perspiration.

Film-forming and/or setting polymers are often used as ingredients in cosmetic agents for temporary deformation of keratin fibers. Such agents include hair sprays, hair waxes, hair gels, setting lotions etc. However, these polymers may also be used in other cosmetic agents such as skin treatment agents.

Cosmetic agents according to the invention preferably comprise agents for treating, in particular, for deforming keratin fibers such as human hair. In this regard, in addition to the task of regulating pheromone release, the film-forming and/or setting polymers have their conventional function of a holding and fixing ingredient.

The above-stated 16-androstene steroids of formula (I) and/or estrene steroids of formula (II) are used as the pheromone.

The R¹ constituent here is an oxo group, meaning an oxygen atom, which at variance with the schematic representation of the linkage of R¹ in formula (I), is attached to the ring system via a double bond, a hydroxyl group, a C₁-C₆ alkoxy, an acyloxy, or a benzoyloxy group. The R² constituent is hydrogen, a hydroxyl group, a C₁-C₆ alkyl, hydroxy-C₁-C₆-alkyl, C₁-C₆-alkoxy-C₁-C₆-alkyl, C₁-C₆ alkoxy, acyl, acyloxy, acyl-C₁-C₆-alkyl or acyloxy-C₁-C₆-alkyl group. Suitable C₁-C₆ alkoxy groups which may be mentioned by way of example include methoxy, ethoxy, propoxy, butoxy, pentoxy and hexyloxy. Suitable acyloxy groups include, in particular, groups derived from their corresponding C₁-C₆ carboxylic acids, for example, formyloxy, acetoxy or propionoxy. Suitable C₁-C₆ alkyl groups include, for example, methyl, ethyl, propyl, isopropyl, butyl, isobutyl, tert.-butyl, pentyl and hexyl. Suitable hydroxy-C₁-C₆-alkyl groups include hydroxymethyl, alpha-hydroxyethyl, beta-hydroxyethyl, 1-hydroxypropyl, 2-hydroxypropyl, 3-hydroxypropyl, 1-hydroxybutyl, 2-hydroxybutyl, 3-hydroxybutyl, 4-hydroxybutyl, 1-hydroxypentyl, 2-hydroxypentyl, 3-hydroxypentyl, 4-hydroxypentyl, 5-hydroxypentyl, 1-hydroxyhexyl, 2-hydroxyhexyl, 3-hydroxyhexyl, 4-hydroxyhexyl, 5-hydroxyhexyl and 6-hydroxyhexyl. Suitable C₁-C₆-alkoxy-C₁-C₆-alkyl groups included methoxymethyl, methoxyethyl, methoxypropyl, methoxybutyl, methoxypentyl, methoxyhexyl, ethoxymethyl, ethoxyethyl, ethoxypropyl, ethoxybutyl, ethoxypentyl, ethoxyhexyl, propoxymethyl, propoxyethyl, propoxypropyl, propoxybutyl, propoxypentyl, propoxyhexyl, butoxymethyl, butoxyethyl, butoxypropyl, butoxybutyl, butoxypentyl, butoxyhexyl, pentoxymethyl, pentoxyethyl, pentoxypropyl, pentoxybutyl, pentoxypentyl, pentoxyhexyl, hexyloxymethyl, hexyloxyethyl, hexyloxypropyl, hexyloxybutyl, hexyloxypentyl and hexyloxyhexyl.

16-androstene steroids according to formula (I) which are preferably used are those whose R¹ constituent is an oxo, hydroxyl, methoxy, acetoxy, propionoxy or benzoyloxy group, and particularly preferably those whose R¹ constituent is an oxo or hydroxyl group.

Preferred 16-androstene steroids according to formula (I) include those whose R² constituent is hydrogen or a hydroxyl, methyl, ethyl, n-propyl, iso-propyl, methoxy or ethoxy group, and particularly preferably those whose R² constituent is hydrogen or a methyl group.

Particularly preferred 16-androstene steroids include those according to formula (I) whose R¹ and R² constituents are the stated preferred or particularly preferred groups.

One very particularly preferred pheromone is 4,16-androstadien-3-one, which is shown in formula (III)—

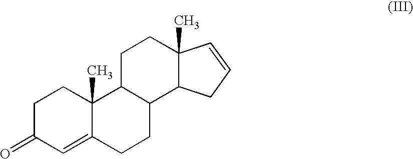

Preferred estrene steroids are compounds according to formula (II) wherein R⁴ is an oxo group, hydroxyl group or hydrogen.

Preferred estrene steroids according to formula (II) also include those wherein R⁵ is hydrogen or a hydroxyl group, and/or wherein R⁶ is hydrogen.

Particularly preferred estrene steroids are compounds according to formula (II) wherein R⁴, R⁵ and R⁶ have the previously indicated preferred meanings. 1,3,5(10)-Estratriene-3,17β-diol, 1,3,5(10)-estratriene-3,16α,17β-triol, 1,3,5(10)-estratrien-3-ol-17-one and 1,3,5(10),16-estratetraen-3-ol may, in particular, be mentioned. 1,3,5(10),16-Estratetraen-3-ol is very particularly preferred.

In a preferred embodiment, agents according to the invention contain a mixture of at least one 16-androstene steroid according to formula (I) and at least one estrene steroid according to formula (II). Particularly preferred are mixtures wherein preferred 16-androstene steroids according to formula (I) and preferred estrene steroids according to formula (II) as previously indicated are used, and even moreso those particularly preferred according to the above explanations are used.

A particularly preferred agent includes a mixture of 4,16-androsta-dien-3-one and 1,3,5(10),16-estratetraen-3-ol.

Agents according to the invention contain the steroid or the steroid mixture in an amount of from 1×10⁻⁷ to 1×10⁻² wt. %, preferably in an amount of from 1×10⁻⁴ to 5×10⁻³ wt. %, and particularly preferably in an amount of from 3×10⁻⁴ to 1×10⁻³ wt. %, in each case relative to the entire cosmetic agent, wherein, in the event that the cosmetic agent contains propellant, the latter is not taken into consideration. If a mixture of various steroids is used, the stated quantities relate to the sum of the steroids used, wherein the quantity ratio of the various steroids to one another may be freely varied.

If the agent contains a mixture of at least one 16-androstene steroid according to formula (I) and at least one estrene steroid according to formula (II), the 16-androstene steroid and estrene steroid are preferably used in quantities such that the weight ratio of the sum of the 16-androstene steroids used to the sum of the estrene steroids used amounts to 1:100 to 100:1, preferably 1:10 to 10:1.

Agents according to the invention also contain 0.1 to 30.0 wt. %, based on weight of the entire agent, of at least one film-forming and/or setting polymer. In the event that the cosmetic agent contains propellant, the latter is not taken into consideration in calculating the corresponding proportions.

The film-forming and/or setting polymer is present in the agent preferably in an amount of from 0.5 to 25.0 wt. %, particularly preferably from 1.0 to 20.0 wt. %, based on weight of the entire agent. A plurality of film-forming and/or setting polymers may also be contained therein.

Useful film-forming and/or setting polymers may be either permanently or temporarily cationic, anionic, nonionic or amphoteric. When at least two film-forming and/or setting polymers are used in the present invention, they may have different charges. For example, it may be preferable according to the invention to use an ionic film-forming and/or setting polymer together with an amphoteric and/or nonionic film-forming and/or setting polymer. Use of at least two oppositely charged film-forming and/or setting polymers is also preferred. In the latter case, a particular embodiment may additionally contain at least one further amphoteric and/or nonionic film-forming and/or setting polymer.

Since polymers are often multifunctional, their functions cannot always be clearly and unambiguously delimited from one another. This is particularly true of film-forming and setting polymers. Some film-forming polymers will nevertheless be described by way of example. It should, however, be pointed out that both film-forming and setting polymers are useful for the purposes of the present invention. Since the two properties are also not wholly mutually independent, the term “setting polymers” should also be understood to include “film-forming polymers” and vice versa.

Preferred properties of film-forming polymers include film formation. Film-forming polymers refers to those polymers which, on drying, leave behind a continuous film on the skin, hair or nails. Such film formers can be used in a variety of cosmetic products, such as face masks, make-up, hair setting preparations, hairsprays, hair gels, hair waxes, hair masks, shampoos or nail polishes. Preferred polymers include those which exhibit sufficient solubility in alcohol or water/alcohol mixtures so as to be present in agents according to the invention in dissolved form. The film-forming polymers may be of synthetic or natural origin.

Film-forming polymers according to the invention further include polymers which, when applied in a 0.01 to 20 wt. % aqueous, alcoholic or aqueous/alcoholic solution, deposit a transparent polymer film on the hair. The film-forming polymers can be anionically, amphoterically, nonionically, permanently cationically or temporarily cationically charged.

Suitable synthetic, film-forming, hair-setting polymers include homo- or copolymers synthesized from at least one of the following monomers: vinylpyrrolidone, vinylcaprolactam, vinyl esters such as for example vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, wherein the alkyl groups of these monomers are preferably C₁ to C₇ alkyl groups, particularly preferably C₁ to C₇ alkyl groups.

Examples include homopolymers of vinylcaprolactam, vinylpyrrolidone or N-vinylformamide. Further suitable synthetic film-forming, hair-setting polymers include copolymers of vinylpyrrolidone and vinyl acetate, terpolymers of vinylpyrrolidone, vinyl acetate and vinyl propionate, polyacrylamides (commercially available under the tradename Akypomine® P 191 from CHEM-Y, Emmerich, or from Seppic under the trade name Sepigel 305®); polyvinyl alcohols, available from, for example, DuPont under the trade name Elvanol® or from Air Products under the trade name Vinol® 523/540; as well as polyethylene glycol/polypropylene glycol copolymers distributed, for example, by Union Carbide under the trade name Ucon®.

Examples of suitable natural film-forming polymers include cellulose derivatives such as hydroxypropyl cellulose with a molecular weight of 30,000 to 50,000 g/mol, commercially available from Lehmann & Voss, Hamburg, Germany under the trade name Nisso SI®.

Setting polymers assist in holding or building up the volume and fullness of the overall hairstyle. These “setting” polymers are also known as film-forming polymers and therefore are commonly used in hair shaping treatment agents such as hair setting preparations, hair mousses, hair waxes, hairsprays and so forth. Film formation may, in this respect, take place only at points and connect only a few fibers together.

Substances which provide the hair with hydrophobic properties are preferred here, because they reduce the tendency of the hair to absorb moisture (i.e., water). This reduces the tendency of hair strands to hang down limply and thus ensures that a hairstyle retains its structure for a long time. The “curl retention” test is often used as a test method in such cases. These polymeric substances may additionally be successfully incorporated into leave-on and rinse-off hair tonics or shampoos. Since polymers are often multifunctional, meaning they have desirable effects in a plurality of applications, many polymers are classified in a plurality of groups according to their mode of action, for example, also in the CTFA Handbook. Because of the significance of the setting polymers, these will be listed below explicitly by their INCI names. This list of polymers preferably used according to the invention also includes the stated film-forming polymers.

Examples of common film-forming/setting polymers include Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylamino-hydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/-Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, PolybetaAlanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Poliquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylamino-ethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, and Yeast Palmitate.

Uniform release of the pheromones and a long-lasting, stable effect may be achieved if the agent contains film-forming and/or setting polymers insensitive to environmental influences, in particular to moisture. Corresponding film-forming and/or setting polymers are accordingly preferably used.

Agents according to the invention preferably contain at least one film-forming and/or setting polymer chosen from
aminomethylpropanol salts of copolymers of allyl methacrylate with one or more monomers chosen from acrylic acid, methacrylic acid, acrylic acid esters and methacrylic acid esters,
vinylpyrrolidone-vinyl acetate copolymers,
vinylpyrrolidone-vinylcaprolactam-dimethylaminopropylacrylarnide copolymers,
copolymers of octylacrylamide with t-butylaminoethyl methacrylate and two or more monomers chosen from acrylic acid, methacrylic acid, acrylic acid esters and methacrylic acid esters, and
copolymers of C₁₋₂ alkyl succinates with hydroxyalkyl acrylates and one or more monomers chosen from acrylic acid, methacrylic acid, acrylic acid esters and methacrylic acid esters.

Corresponding film-forming and/or setting polymers are commercially obtainable.

Agents according to the invention preferably contain as the film-forming and/or setting polymer an aminomethylpropanol salt of a copolymer of allyl methacrylate with one or more monomers chosen from acrylic acid, methacrylic acid, acrylic acid esters and methacrylic acid esters.

The stated acrylic acid esters and methacrylic acid esters preferably comprise C₁-C₁₂ alkyl acrylates and C₁-C₁₂ alkyl methacrylates, particularly preferably methyl acrylate, ethyl acrylate, propyl acrylate, methyl methacrylate, ethyl methacrylate, propyl methacrylate and mixtures thereof.

The aminomethylpropanol salt of copolymers of allyl methacrylate with one or more monomers chosen from acrylic acid, methacrylic acid, acrylic acid esters and methacrylic acid esters is preferably the copolymer with the INCI name AMP-Acrylates/Allyl Methacrylate Copolymer, distributed by Noveon under the trade name Fixate™ G-100. Agents according to the invention very particularly preferably contain this copolymer.

A preferred vinylpyrrolidone-vinyl acetate copolymer is the PVP/VA copolymer 60-40 W (INCI name: VPA/A Copolymer, Aqua, Laurtrimonium Chloride).

A vinylpyrrolidone-vinylcaprolactam-dimethylaminopropylacrylamide copolymer preferably used is the copolymer with the INCI name VP/Vinyl Caprolactam/DMAPA Acrylates Copolymer, obtainable from ISP under the name Aquaflex SF 40.

A preferred copolymer of octylacrylamide with t-butylaminoethyl methacrylate and two or more monomers chosen from acrylic acid, methacrylic acid, acrylic acid esters and methacrylic acid esters is the copolymer with the INCI name Octylacrylamide/Acrylates Butylaminoethyl Methacrylates Copolymer, obtainable from National Starch under the name Amphomer®.

A preferred copolymer of C₁₋₂ alkyl succinates with hydroxyalkyl acrylates and one or more monomers chosen from acrylic acid, methacrylic acid, acrylic acid esters and methacrylic acid esters is the copolymer with the INCI name Acrylates/C1-2 Succinates/Hydroxyacrylates Copolymer, obtainable from ISP under the name Allianz™ LT 120.

The agents according to the invention may furthermore contain any active ingredients, additives and auxiliary substances known for such agents. Conditioners are of particular significance in this connection.

It has been found that the addition of a silicone component has a particularly positive impact on the uniform release of the pheromone and a long-lasting, stable effect on this component.

Agents according to the invention therefore preferably contain at least one silicone oil and/or silicone gum.

Silicone oils or silicone gums suitable according to the invention include dialkyl- and alkylarylsiloxanes such as dimethylpolysiloxane and methylphenylpolysiloxane, as well as the alkoxylated, quaternized or anionic derivatives thereof. Preference is given to cyclic and linear polydialkylsiloxanes, alkoxylated and/or aminated derivatives thereof, dihydroxypolydimethylsiloxanes and polyphenylalkylsiloxanes.

Silicone oils bring about a variety of effects. For example, they simultaneously influence dry and wet combability, the feel of dry and wet hair, and its gloss. The term silicone oil is understood by a person skilled in the art to refer to a variety of organo-silicon compounds of different structures. The first among these are the dimethiconols (S1). These may be both linear and branched and cyclic or cyclic and branched. Linear dimethiconols may be represented by the following structural formula (S1-I)—

(HOSiR¹₂)—O—(SiR²₂—O—)ₓ—(SiR¹₂OH) (S1-I)

Branched dimethiconols may be represented by the structural formula (S1-II)—

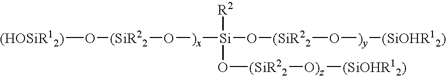

The R¹ and R² constituents are mutually independent and represent in each case hydrogen, a methyl residue, a C₂ to C₃₀ linear, saturated or unsaturated hydrocarbon residue, a phenyl residue and/or an aryl residue. Non-limiting examples of residues represented by R¹ and R² include alkyl residues such as methyl, ethyl, propyl, isopropyl, butyl, isobutyl, pentyl, isopentyl, neopentyl, amyl, isoamyl, hexyl, isohexyl and the like; alkenyl residues such as vinyl, halovinyl, alkylvinyl, allyl, haloallyl, alkylallyl and the like; cycloalkyl residues such as cyclobutyl, cyclopentyl, cyclohexyl and the like; phenyl residues; benzyl residues; halogenated hydrocarbon residues such as 3-chloropropyl, 4-bromobutyl, 3,3,3-trifluoropropyl, chlorocyclohexyl, bromophenyl, chlorophenyl and the like; and sulfur-containing residues such as mercaptoethyl, mercaptopropyl, mercaptohexyl, mercaptophenyl and the like. R¹ and R² are preferably an alkyl residue having approximately 1 to 6 carbon atoms, and R¹ and R² are particularly preferably methyl. The numbers x, y and z are mutually independent integers and run in each case from 0 to 50,000. Molar weights of the dimethiconols are between 1000 D and 10000000 D. Viscosities are between 100 and 10000000 cPs measured at 25° C. with a glass capillary viscometer using the Dow Corning Corporate Test Method CTM 0004 of 20 Jul. 1970. Preferred viscosities are between 1000 and 5000000 cPs; and very particularly preferred viscosities are between 10000 and 3000000 cPs. The most preferred range is between 50000 and 2000000 cPs.

The following commercial products are examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all above-stated from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all above-stated from Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all above-stated from Crompton Corporation), SM555, SM2725, SM2765, SM2785 (all above-stated from GE Silicones), Taylor T-Sil CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all above-stated from Wacker-Chemie GmbH).

Dimethicones (S2) form the second group of silicones which may be present according to the invention. These may be both linear and branched and cyclic or cyclic and branched. Linear dimethicones may be represented by the following structural formula (S2-I)—

(SiR¹₃)—O—(SiR¹R²—O—)ₓ—(SiR¹₃) (S2-I)

Branched dimethicones may be represented by the structural formula (S2-II)—

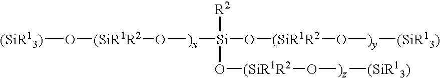

The R¹ and R² constituents are mutually independent and represent in each case hydrogen, a methyl residue, a C₂ to C₃₀ linear, saturated or unsaturated hydrocarbon residue, a phenyl residue and/or an aryl residue. Non-limiting examples of residues represented by R¹ and R² include alkyl residues such as methyl, ethyl, propyl, isopropyl, butyl, isobutyl, pentyl, isopentyl, neopentyl, amyl, isoamyl, hexyl, isohexyl and the like; alkenyl residues such as vinyl, halovinyl, alkylvinyl, allyl, haloallyl, alkylallyl and the like; cycloalkyl residues such as cyclobutyl, cyclopentyl, cyclohexyl and the like; phenyl residues; benzyl residues; halogenated hydrocarbon residues such as 3-chloropropyl, 4-bromobutyl, 3,3,3-trifluoropropyl, chlorocyclohexyl, bromophenyl, chlorophenyl and the like; and sulfur-containing residues such as mercaptoethyl, mercaptopropyl, mercaptohexyl, mercaptophenyl and the like. R¹ and R² are preferably an alkyl residue containing approximately 1 to 6 carbon atoms, and R¹ and R² are particularly preferably methyl. The numbers x, y and z are mutually independent integers and run in each case from 0 to 50,000. Molar weights of the dimethicones are between 1000 D and 10000000D. Viscosities are between 100 and 10000000 cPs measured at 25° C. with a glass capillary viscometer using the Dow Corning Corporate Test Method CTM 0004 of 20 Jul. 1970. Preferred viscosities are between 1000 and 5000000 cPs, and very particularly preferred viscosities are between 10000 and 3000000 cPs. Viscosity is very particularly preferably in the range between 50000 and 2000000 cPs.

Dimethicone copolyols (S3) form a further group of silicones which are suitable. Dimethicone copolyols may be represented by the following structural formulae

(SiR¹₃)—O—(SiR²₂—O—)ₓ—(SiR²PE—O—)_{y}—(SiR¹₃) (S3-I),

PE—(SiR¹₂)—O—(SiR²₂—O—)ₓ—(SiR¹₂)—PE (S3-II)

Branched dimethicone copolyols may be represented by the structural formula (S3-III)—

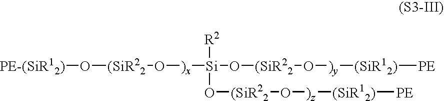

or by the structural formula (S3-IV)—

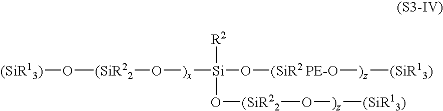

The R¹ and R² constituents are mutually independent and represent in each case hydrogen, a methyl residue, a C₂ to C₃₀ linear, saturated or unsaturated hydrocarbon residue, a phenyl residue and/or an aryl residue. Non-limiting examples of residues represented by R¹ and R² include alkyl residues such as methyl, ethyl, propyl, isopropyl, butyl, isobutyl, pentyl, isopentyl, neopentyl, amyl, isoamyl, hexyl, isohexyl and the like; alkenyl residues such as vinyl, halovinyl, alkylvinyl, allyl, haloallyl, alkylallyl and the like; cycloalkyl residues such as cyclobutyl, cyclopentyl, cyclohexyl and the like; phenyl residues; benzyl residues; halogenated hydrocarbon residues such as 3-chloropropyl, 4-bromobutyl, 3,3,3-trifluoropropyl, chlorocyclohexyl, bromophenyl, chlorophenyl and the like; and sulfur-containing residues such as mercaptoethyl, mercaptopropyl, mercaptohexyl, mercaptophenyl and the like. R¹ and R² are preferably an alkyl residue containing approximately 1 to 6 carbon atoms, and R¹ and R² are particularly preferably methyl. PE denotes a polyoxyalkylene residue. Preferred polyoxyalkylene residues are derived from ethylene oxide, propylene oxide and glycerol. The numbers x, y and z are mutually independent integers and run in each case from 0 to 50,000. The molar weights of the dimethicones are between 1000 D and 10000000 D. Viscosities are between 100 and 10000000 cPs measured at 25° C. with a glass capillary viscometer using the Dow Corning Corporate Test Method CTM 0004 of 20 Jul. 1970. Preferred viscosities are between 1000 and 5000000 cPs; and very particularly preferred viscosities are between 10000 and 3000000 cPs. The most preferred range is between 50000 and 2000000 cPs.

Corresponding dimethicone copolyols are commercially obtainable and are distributed, for example, by Dow Corning under the name Dow Corning® 5330 Fluid.

Dimethiconols, dimethicones and/or dimethicone copolymers may, of course, also already assume emulsion form. In this case, the corresponding emulsion of dimethiconols, dimethicones and/or dimethicone copolyols may be produced both after the production of the corresponding dimethiconols, dimethicones and/or dimethicone copolyols from the latter, using conventional methods of emulsification known to a person skilled in the art. To this end, any cationic, anionic, nonionic or zwitterionic surfactants and emulsifiers may be used as auxiliary materials for producing the corresponding emulsions. Emulsions of the dimethiconols, dimethicones and/or dimethicone copolyols may, of course, be also produced directly by an emulsion polymerization method. Such methods are also well known to a person skilled in the art. In this respect, reference is made, for example, to the “Encyclopedia of Polymer Science and Engineering”, Vol. 15, 2^{nd} Ed., pp. 204-308, John Wiley & Sons., Inc. (1989). Reference is explicitly made to this standard work.

If the dimethiconols, dimethicones and/or dimethicone copolyols are used as an emulsion, droplet size of emulsified particles according to the invention can be from to 0.01 to 10000 μm, preferably from 0.01 to 100 μm, particularly preferably from 0.01 to 20 μm and very particularly preferably from 0.01 to 10 μm. Particle size is determined using the light scattering method.

If branched dimethiconols, dimethicones and/or dimethicone copolyols are used, it should be understood that branching is greater here than in the chance branching which occurs due to impurities in the respective monomers. For the purposes of the present invention, branched dimethiconols, dimethicones and/or dimethicone copolyols refer to those having a degree of branching greater than 0.01%. Preferably, the degree of branching is greater than 0.1%, and very particularly preferably greater than 0.5%. The degree of branching is determined based on the ratio of unbranched monomers to branching monomers (i.e., the quantity of tri- and tetrafunctional siloxanes). According to the invention, dimethiconols, dimethicones and/or dimethicone copolyols having both a low and high degree of branching may be very particularly preferred.

Suitable silicones further include amino-functional silicones (S4), in particular those silicones designated by the INCI name Amodimethicone. These include silicones having at least one, optionally substituted, amino group.

Such silicones may, for example, be described by the formula (S4-I)—

M(RₐQ_{b}SiO_{(4-a-b)/2})ₓ(R_{c}SiO_{(4-c)/2)})_{y}M (S4-I)

wherein in the above formula R is a hydrocarbon or a hydrocarbon residue with approximately 1 to approximately 6 carbon atoms; Q is a polar residue of the general formula —R¹Z, in which R¹ is a divalent linking group attached to hydrogen and Z is an organic, amino-functional residue containing at least one amino-functional group and composed of carbon and hydrogen atoms, carbon, hydrogen and oxygen atoms or carbon, hydrogen and nitrogen atoms; “a” assumes values in the range from approximately 0 to approximately 2, “b” assumes values in the range from approximately 1 to approximately 3, “a”+“b” is less than or equal to 3, and “c” is a number from approximately 1 to approximately 3; x is a number from approximately 1 to approximately 2,000, preferably from approximately 3 to approximately 50 and most preferably from approximately 3 to approximately 25; y is a number from approximately 20 to approximately 10,000, preferably from approximately 125 to approximately 10,000 and most preferably from approximately 150 to approximately 1,000; and M is a suitable silicone end group as known in the prior art, preferably trimethylsiloxy. Non-limiting examples of residues represented by R include alkyl residues such as methyl, ethyl, propyl, isopropyl, butyl, isobutyl, amyl, isoamyl, hexyl, isohexyl and the like; alkenyl residues such as vinyl, halovinyl, alkylvinyl, allyl, haloallyl, alkylallyl and the like; cycloalkyl residues such as cyclobutyl, cyclopentyl, cyclohexyl and the like; phenyl residues; benzyl residues; halogenated hydrocarbon residues such as 3-chloropropyl, 4-bromobutyl, 3,3,3-trifluoropropyl, chlorocyclohexyl, bromophenyl, chlorophenyl and the like; and sulfur-containing residues such as mercaptoethyl, mercaptopropyl, mercaptohexyl, mercaptophenyl and the like. R is preferably an alkyl residue containing approximately 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄— and —(CH₂)₃C(O)SCH₂CH₂—.

Z is an organic, amino-functional residue containing at least one functional amino group. One possible formula for Z is NH(CH₂)_{z}NH₂ wherein z is an integer from 1 to 50. Another possible formula for said Z is —NH(CH₂)_{z}NH(CH₂)_{zz}, wherein both z and zz are mutually independent and are an integer from 1 to 50, and wherein this structure comprises diamino ring structures such as piperazinyl. Z is particularly preferably an —NHCH₂CH₂NH₂ residue. Another possible formula for Z is —(CH₂)_{z}NX¹X² or —NX¹X², in which X¹ and X² are in each case mutually independently selected from hydrogen and a hydrocarbon residue with approximately 1 to approximately 6 carbon atoms.

Q very particularly preferably denotes a polar, amino-functional residue of the formula —CH₂CH₂CH₂NHCH₂CH₂NH₂.

The molar ratio of the RₐQ_{B}SiO_{(4-a-b)/2} units to the R_{c}SiO_{(4-c)/2} units is in the range from approximately 1:2 to approximately 1:65, preferably from approximately 1:5 to approximately 1:65, and particularly preferably from approximately 1:15 to approximately 1:20. If one or more silicones of the above formula are used, then the various variable substituents in the above formula may differ in the various silicone components which may be present in the silicone mixture.

Preferred amino-functional silicones are of the formula (S4-II)—

R′ₐG₃₋ₐ-Si(OSiG₂)ₙ—(OSiG_{b}R′_{2-b})ₘ—O—SiG₃₋ₐ-R′ₐ (S4-II),

wherein
G is hydrogen, a phenyl group, —OH, —O—CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, or —C(CH₃)₃;
a is a number between 0 and 3, in particular 0;
b is a number between 0 and 1, in particular 1,
m and n are numbers, the sum of which (m+n) amounts to between 1 and 2000, preferably between 50 and 150, with n preferably a value from 0 to 1999, and in particular from 49 to 149, and m preferably a value from 1 to 2000, in particular from 1 to 10,
R′ is a monovalent residue chosen from
   —N(R″)—CH₂—CH₂—N(R″)₂
   —N(R″)₂
   —N⁺(R″)₃A⁻
   —N⁺H(R″)₂A⁻
   —N⁺H₂(R″)A⁻
   N(R″)—CH₂—CH₂—N⁺R″H₂A⁻,
wherein each R″ is identical or different residue from the group —H, phenyl, benzyl, C₁₋₂₀ alkyl residues, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, or —C(CH₃)₃, and A⁻ represents an anion preferably chosen from chloride, bromide, iodide or methosulfate.

Particularly preferred amino-functional silicones are of the formula (S4-III)—

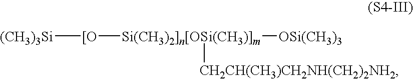

These silicones are known under their INCI Designation as Trimethylsilylamodimethicones.

Particularly preferred amino-functional silicones are those of the formula (S4-IV)

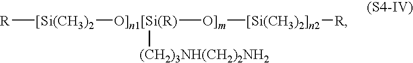

These silicones are known under their INCI Designation as Amodimethicones and can be obtained, for example, in the form of an emulsion available as the commercial product Dow Corning® 949 in a mixture with a cationic and a nonionic surfactant.

Preferably useful amino-functional silicones include those having an amine value greater than 0.25 meq/g, preferably greater than 0.3 meq/g, and particularly preferably greater than 0.4 meq/g. The amine value here is milliequivalents of amine per gram of amino-functional silicone. It can be determined by titration and may also be stated in the unit mg of KOH/g.

Further suitable silicones include, for example—
oligomeric polydimethylcyclosiloxanes (INCI name: Cyclomethicone), in particular the tetrameric and pentameric compounds commercially distributed by Dow Corning as DC 245 Fluid, DC 344 or DC 345,
hexamethyl-disiloxane (INCI name: Hexamethyldisiloxane), for example, the product distributed under the name Abil® K 520,
polyphenylmethylsiloxanes (INCI name: Phenyl Trimethicone), for example, the commercial product DC 556 Cosmetic Grade Fluid from Dow Corning,
esters and partial esters of silicone-glycol copolymers, as are, for example, distributed by Fanning under the tradename Fancorsil® LIM (INCI name: Dimethicone Copolyol Meadowfoamate),
anionic silicone oils, such as for example the product Dow Corning® 1784.

In a preferred embodiment, agents according to the invention contain at least two different silicone derivatives, particularly preferably a combination of a volatile and a non-volatile silicone. For the purposes of the invention, volatile silicones include those which exhibit a volatility greater than or equal to the volatility of cyclic, pentameric dimethylsiloxane. Such combinations are also obtainable commercially (for example Dow Corning® 1401, Dow Corning® 1403 and Dow Corning® 1501, each being mixtures of a cyclomethicone and a dimethiconol).

Preferred mixtures of different silicones are for example dimethicones and dimethiconols, linear dimethicones and cyclic dimethiconols. One very particularly preferred mixture of silicones consists of at least one cyclic dimethiconol and/or dimethicone, at least one further non-cyclic dimethicone and/or dimethiconol and at least one amino-functional silicone.

If different silicones are used, the mixing ratio is extensively variable. However, all silicones used for the mixture are preferably used in a ratio of from 5:1 to 1:5 in the case of a binary mixture. A ratio of 3:1 to 1:3 is particularly preferable. Very particularly preferred mixtures contain silicones broadly in a ratio of approx. 1:1, in each case relative to the quantities used in wt. %.

Agents contain silicones preferably in quantities of 1-25 wt. %, particularly preferably of 5-20 wt. % and particularly preferably of 7-15 wt. %, based on total weight of the agent.

It has also proved advantageous for the agent according to the invention to contain as conditioner at least one protein hydrolyzate and/or a derivative thereof.

Protein hydrolyzates are product mixtures obtained by acidically, basically or enzymatically catalyzed degradation of proteins. According to the invention, the term protein hydrolyzate also covers total hydrolyzates and individual amino acids and their derivatives, as well as mixtures of different amino acids. Furthermore, polymers built up from amino acids and amino acid derivatives are also covered according to the invention by the term protein hydrolyzates. The latter include, for example, polyalanine, polyasparagine, polyserine etc. Further examples of compounds which may be used according to the invention are L-alanyl-L-proline, polyglycine, glycyl-L-glutamine or D/L-methionine-5-methylsulfonium chloride. It goes without saying that, according to the invention, β-amino acids and their derivatives (e.g., β-alanine, anthranilic acid or hippuric acid) may also be used. The molecular weight of useful protein hydrolyzates according to the invention is from about 75, the molecular weight of glycine, to 200,000, the molecular weight preferably from about 75 to about 50,000 and very particularly preferably from about 75 to about 20,000 Daltons.

Protein hydrolyzates of both plant and animal origin or marine or synthetic origin may be used according to the invention.

Animal protein hydrolyzates include elastin, collagen, keratin, silk and milk protein hydrolyzates and their salt form. Such products are distributed, for example, under the trademark Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda). Use of silk protein hydrolyzates is of particular interest.

Protein hydrolyzates of various plant origins such as soy, almond, pea, potato and wheat are, for example, obtainable under the trademark Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although use of protein hydrolyzates as such is preferred, amino acid mixtures obtained in other ways may also optionally be used instead. It is likewise possible to use derivatives of protein hydrolyzates, for example, the fatty acid condensation products thereof. Such products are distributed, for example, under the names Lamepon® (Cognis), Lexein® (Inolex), Crolastin® (Croda), Crosilk® (Croda) or Crotein® (Croda).

Hydrolyzates according to the invention include all isomeric forms such as cis-trans isomers, diastereomers and chiral isomers.

According to the invention, it is also possible to use a mixture of a plurality of protein hydrolyzates.

When agents according to the invention contain protein hydrolyzates, they can be used, for example, in amounts from 0.01 wt. % to 20 wt. %, preferably from 0.05 wt. % to 15 wt. % and very particularly preferably in quantities from 0.05 wt. % to 5 wt. %, in each case based on the total ready-to-use preparation.

Preferably used conditioners include the silicones and/or protein hydrolyzates just mentioned, since using them has a particularly advantageous effect on the release and stability of the pheromones. It is also possible to add other conditioners to the agents.

For example, agents according to the invention may include at least one cationic surfactant as a conditioner from another class of compounds.

According to the invention, preference is given to cationic surfactants such as quaternary ammonium compounds, ester quats and amidoamines. Preferred quaternary ammonium compounds include ammonium halides, in particular chlorides and bromides such as alkyltrimethylammonium chlorides, dialkyldimethylammonium chlorides and trialkylmethylammonium chlorides, for example, cetyltrimethylammonium chloride, stearyltrimethylammonium chloride, distearyldimethylammonium chloride, lauryldimethyl-ammonium chloride, lauryldimethylbenzylammonium chloride and tricetylmethylammonium chloride, as well as imidazolinium compounds known under the INCI names Quaternium 27 and Quaternium 83. Long alkyl chains of the above-stated surfactants preferably comprise 10 to 18 carbon atoms.

Ester quats are known substances containing both at least one ester function and at least one quaternary ammonium group as a structural element. Preferred ester quats include quaternized ester salts of fatty acids with triethanolamine, quaternized ester salts of fatty acids with diethanolalkylamines and quaternized ester salts of fatty acids with 1,2-dihydroxy-propyldialkylamines. Such products are distributed, for example, under the tradenames Stepantex®, Dehyquart® and Armocare®. The products Armocare® VGH-70, an N,N-bis(2-palmitoyloxyethyDdimethylammonium chloride, and Dehyquart® F-75, Dehyquart® C-4046, Dehyquart® L80 and Dehyquart® AU-35 are examples of such ester quats.

Alkylamidoamines are conventionally produced by amidating natural or synthetic fatty acids and fatty acid cuts with dialkylaminoamines. One compound from this group of substances particularly suitable according to the invention is stearamidopropyldimethylamine, commercially available under the name Tegoamid® S18.

Agents according to the invention preferably contain cationic surfactants in quantities of from 0.05 to 10 wt. %, relative to the total ready-to-use preparation. Quantities of 0.1 to 5 wt. % are particularly preferred.

Conditioning polymers are likewise suitable as a conditioner.

A first group of conditioning polymers comprises cationic polymers. Cationic polymers are polymers having a group in the main and/or side chain which may be “temporarily” or “permanently” cationic. Polymers which are designated “permanently cationic” according to the invention are those which, irrespective of the pH value of the agent, comprise a cationic group. As a rule, these are polymers which contain a quaternary nitrogen atom, for example, in the form of an ammonium group. Preferred cationic groups are quaternary ammonium groups. Polymers which have proven particularly suitable are in particular those in which the quaternary ammonium group is bound via a C₁₋₄ hydrocarbon group to a main polymer chain synthesized from acrylic acid, methacrylic acid or the derivatives thereof.

Homopolymers of the general formula (G1-I)—

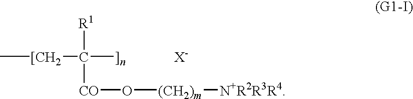

wherein R¹ is —H or —CH₃; R², R³ and R⁴ are mutually independently chosen from C₁₋₄ alkyl, alkenyl or hydroxyalkyl groups; m=1, 2, 3 or 4; n is a natural number; and X⁻ a physiologically acceptable organic or inorganic anion. Cationic copolymers consisting substantially of the monomer units listed in formula (G1-I) and nonionogenic monomer units are particularly preferred. In the context of these polymers, those which are preferred according to the invention are those for which at least one of the following conditions applies:

R¹ is a methyl group;

R², R³ and R⁴ are methyl groups; and

m has the value 2.

Physiologically acceptable counterions X⁻ include halide ions, sulfate ions, phosphate ions, methosulfate ions and organic ions such as lactate, citrate, tartrate and acetate ions. Halide ions, in particular chloride, are preferred.

A particularly suitable homopolymer is poly(methacryloyloxyethyltrimethylammonium chloride) with the INCI name Polyquaternium 37, and which can be crosslinked. Crosslinking can be initiated with olefinically polyunsaturated compounds such as divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion having a polymer fraction of no less than 30 wt. %. Such polymer dispersions are commercially available under the names Salcare® SC 95 (approx. 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Copolymers with monomer units according to the formula (G1-I) preferably contain acrylamide, methacrylamide, acrylic acid C₁₋₄ alkyl esters and methacrylic acid C₁₋₄ alkyl esters as nonionogenic monomer units. Acrylamide is particularly preferred among these nonionogenic monomers. These copolymers, like the homopolymers mentioned previously, may also be crosslinked. A copolymer which is preferred according to the invention is crosslinked acrylamide-methacryloyloxyethyltrimethylammonium chloride copolymer. Such copolymers, in which the monomers are present in a weight ratio of approx 20:80, are commercially available as approx. 50% nonaqueous polymer dispersions under the name Salcare® SC 92.

Further preferred cationic polymers include, for example—
quaternized cellulose derivatives, commercially available under the names Celquat® and Polymer JR®. The compounds Celquat® H 100, Celquat® L 200 and Polymer JR® 400 are preferred quaternized cellulose derivatives,
cationic alkyl polyglycosides according to DE-PS 44 13 686,
cationized honey, for example, the commercial product Honeyquat® 50,
cationic guar derivatives, such as the products distributed under the trade names Cosmedia® Guar and Jaguar®,
polysiloxanes with quaternary groups, such as the commercially obtainable products Q2-7224 (manufacturer: Dow Corning; a stabilized trimethylsilylamodimethicone), Dow Corning® 929 Emulsion (containing a hydroxylamine-modified silicone which is also designated an amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, Quaternium 80),
polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as for example vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are offered for sale under the names Luviquat FC 370, FC 550, FC 905 and HM 552,
quaternized polyvinyl alcohol,
and the polymers known by the names Polyquaternium 2, Polyquaternium 17, Polyquaternium 18 and Polyquaternium 27 with quaternary nitrogen atoms in the polymer main chain.

The polymers known under the names Polyquaternium 24 (commercial product, for example, Quatrisoft® LM 200) may also be used as cationic polymers. Copolymers of vinylpyrrolidone, available as commercial products Copolymer 845 (manufacturer: ISP), Gaffix® VC 713 (manufacturer: ISP), Gafquat® ASCP 1011, Gafquat® HS110, Luviquat® 8155 and Luviquat® MS 370, may likewise be used according to the invention.

Further cationic polymers usable according to the invention are “temporarily cationic” polymers. These polymers conventionally contain an amino group which at specific pH values assumes the form of a quaternary ammonium group and is thus cationic. Chitosan and its derivatives are preferred, commercially available, for example, under the trade names Hydagen® CMF, Hydagen® HCMF, Kytamer® PC and Chitolam® NB/101.

Cationic polymers preferably used according to the invention include cationic cellulose derivatives and chitosan and its derivatives, in particular, the commercial products Polymer® JR 400, Hydagen® HCMF and Kytamer® PC, cationic guar derivatives, cationic honey derivatives, in particular, the commercial product Honeyquat® 50, cationic alkyl polyglycosides according to DE-PS 44 13 686 and polymers of the Polyquaternium 37 type.

Cationic polymers additionally include protein hydrolyzates, wherein the underlying protein hydrolyzate may originate from animals (e.g., from collagen, milk or keratin), from plants (e.g., from wheat, maize, rice, potatoes, soy or almonds), from marine life forms (e.g., from fish collagen or algae), or biotechnologically obtained protein hydrolyzates. Protein hydrolyzates underlying the cationic derivatives according to the invention may be obtained from the corresponding proteins by chemical hydrolysis, in particular alkaline or acidic, by enzymatic hydrolysis and/or by a combination of both types of hydrolysis. Protein hydrolysis as a rule gives rise to a protein hydrolyzate with a molecular weight distribution of approx. 100 Daltons up to several thousand Daltons. Cationic protein hydrolyzates whose underlying protein fraction has a molecular weight of 100 up to 25000 Daltons are preferred, more particularly 250 to 5000 Daltons. Cationic protein hydrolyzates also include quaternized amino acids and mixtures thereof. Quaternization of the protein hydrolyzates or of the amino acids is often performed by means of quaternary ammonium salts such as N,N-dimethyl-N-(n-alkyl)-N-(2-hydroxy-3-chloro-n-propyl)-ammonium halides. The cationic protein hydrolyzates may also be still further derivatized. Typical examples of cationic protein hydrolyzates and derivatives according to the invention include those commercially obtainable and mentioned under the INCI names in the International Cosmetic Ingredient Dictionary and Handbook, 7^{th} Ed., (1997) ‘The Cosmetic, Toiletry, and Fragrance Association’, 1101 17^{th} Street, N.W., Suite 300, Washington, D.C. 20036-4702. These include Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, and Quaternium-79 Hydrolyzed Wheat Protein.

Plant-based cationic protein hydrolyzates and derivatives are very particularly preferred.

Amphoteric polymers particularly useful include those polymers substantially composed of

(a) monomers with quaternary ammonium groups according to the general formula (II)—

R¹—CH═CR²—CO—Z—(CₙH₂ₙ)—N⁽⁺⁾R³R⁴R⁵A⁽⁻⁾ (II)
wherein R¹ and R² mutually independently are hydrogen or a methyl group; R³, R⁴ and R⁵ mutually independently are alkyl groups having 1 to 4 carbon atoms; Z is an NH group or an oxygen atom; n is an integer from 2 to 5; and A⁽⁻⁾ is the anion of an organic or inorganic acid, and
   (b) monomeric carboxylic acids according to the general formula (III)—

R⁶—CH═CR⁷—COOH (III)

wherein R⁶ and R⁷ mutually independently are hydrogen or a methyl group.

These compounds may be used according to the invention both directly and in salt form, which is obtained by neutralization of the polymers, for example, with an alkali metal hydroxide. Very particularly preferred polymers are those in which monomers of type (a) are used, in which R³, R⁴ and R⁵ are methyl groups, Z is an NH group and A⁽⁻⁾ is a halide, methoxysulfate or ethoxysulfate ion. Acrylamidopropyltrimethylammonium chloride is a particularly preferred monomer (a). Acrylic acid is preferably used as monomer (b) for the stated polymers.

Agents according to the invention preferably contain conditioning, cationic polymers in an amount of from 0.01 to 5 wt. %, in particular in an amount of from 0.1 to 2 wt. %, in each case based on total weight of the ready-to-use preparation.

It should be noted in this connection that some of the stated conditioning polymers also exhibit film-forming and/or setting characteristics and may therefore also be used as film-forming and/or setting polymers.

Agents according to the invention may also contain at least one vitamin, provitamin, vitamin precursor and/or one of the derivatives thereof as a conditioner.

Preferred vitamins, provitamins and vitamin precursors according to the invention include those conventionally assigned to groups A, B, C, E, F and H.

The group of substances designated as vitamin A includes retinol (vitamin A₁) and 3,4-didehydroretinol (vitamin A₂). β-Carotene is the provitamin of retinol. Examples of substances useful as the vitamin A component include vitamin A acid and its esters, vitamin A aldehyde, and vitamin A alcohol and its esters such as palmitate and acetate. The vitamin A component is preferably used in an amount of from 0.05-1 wt. %, based on total weight of the ready-to-use preparation.

The vitamin B group or the vitamin B complex includes, inter alia
vitamin B₁ (thiamin)
vitamin B₂ (riboflavin)
vitamin B₃. This designation is frequently used for the compounds nicotinic acid and nicotinamide (niacinamide). Nicotinamide is preferred according to the invention and is preferably contained in the agents according to the invention in quantities of from 0.05 to 1 wt. %, based on total weight of the ready-to-use preparation.
vitamin B₅ (pantothenic acid, panthenol and pantolactone). In the context of this group, panthenol and/or pantolactone are preferably used. Derivatives of panthenol which may be used according to the invention are in particular the esters and ethers of panthenol and cationically derivatized panthenols. Individual representatives include panthenol triacetate, panthenol monoethyl ether and the monoacetate thereof, as well as those panthenol derivatives disclosed in WO 92/13829. Vitamin B₅ compounds are preferably present in agents according to the invention in quantities of from 0.05-10 wt. %, based on total weight of the ready-to-use preparation. Quantities of 0.1-5 wt. % are particularly preferred.
vitamin B₆ (pyridoxine as well as pyridoxamine and pyridoxal). Vitamin B₆ compounds are preferably present in agents according to the invention in quantities of from 0.01-5 wt. %, based on total weight of the ready-to-use preparation. Quantities of 0.05-1 wt. % are particularly preferred.

Vitamin C (ascorbic acid). Vitamin C is preferably used in the agents used according to the invention in quantities of from 0.1 to 3 wt. %, based on total weight of the ready-to-use preparation. Use in the form of a palmitic acid ester, glucosides or phosphates may be preferred. Use in combination with tocopherols may likewise be preferred.

Vitamin E (tocopherols, in particular α-tocopherol). Tocopherol and its derivatives, which include esters such as acetate, nicotinate, phosphate and succinate, are preferably contained in the agents according to the invention in quantities of from 0.05-1 wt. %, based on total weight of the ready-to-use preparation.

Vitamin F. The term “vitamin F” is conventionally understood to refer to essential fatty acids, in particular linoleic acid, linolenic acid and arachidonic acid.

Vitamin H. Vitamin H denotes the compound (3aS,4S,6aR)-2-oxohexahydrothienol[3,4-d]-imidazole-4-valeric acid, more commonly known by the name biotin. Biotin is preferably contained in agents according to the invention in quantities of from 0.0001 to 1.0 wt. %, in particular in quantities of from 0.001 to 0.01 wt %, based on total weight of the ready-to-use preparation.

Agents according to the invention preferably contain vitamins, provitamins and vitamin precursors from groups A, B, C, E and H.

Panthenol, pantolactone, pyridoxine and the derivatives thereof together with nicotinamide and biotin are particularly preferred.

D-panthenol, optionally in combination with at least one of the above-stated silicone derivatives, is very particularly preferably used as a conditioner.

Agents according to the invention may furthermore contain at least one plant extract as conditioner.

Conventionally, these extracts are produced by extraction of the entire plant. However, in individual cases it may also be preferable to produce the extracts solely from the blossoms and/or leaves of the plant.

With regard to plant extracts preferred according to the invention, reference is made in particular to extracts listed in the table starting on page 44 of the 3^{rd} Ed. of the “Leitfaden zur Inhaltsstoffdeklaration kosmetischer Mittel” [“Guidelines for the nomenclature of ingredients in cosmetic agents”], published by the German Cosmetic, Toiletry, Perfumery and Detergent Association (IKW), Frankfurt.

According to the invention, preference is given to extracts from green tea, oak bark, stinging nettle, witch hazel, hops, henna, chamomile, burdock root, horsetail, hawthorn, lime blossom, almond, aloe vera, pine-needle, horse chestnut, sandalwood, juniper, coconut, mango, apricot, lime, wheat, kiwi fruit, melon, orange, grapefruit, sage, rosemary, birch, mallow, lady's smock, wild thyme, yarrow, thyme, melissa, restharrow, coltsfoot, marsh mallow, meristem, ginseng and ginger root.

Particular preference is given to extracts from green tea, oak bark, stinging nettle, witch hazel, hops, chamomile, burdock root, horsetail, lime blossom, almond, aloe vera, coconut, mango, apricot, lime, wheat, kiwi fruit, melon, orange, grapefruit, sage, rosemary, birch, lady's smock, wild thyme, yarrow, restharrow, meristem, ginseng and ginger root.

Very particularly suitable extracts include those from green tea, almond, aloe vera, coconut, mango, apricot, lime, wheat, kiwi fruit and melon.

Extracting agents for producing these plant extracts include water, alcohols and mixtures thereof. Preferred alcohols include lower alcohols such as ethanol and isopropanol, particularly polyhydric alcohols such as ethylene glycol and propylene glycol, both as sole extracting agent and in a mixture with water. Plant extracts based on water/propylene glycol in the ratio 1:10 to 10:1 have proven particularly suitable.

Plant extracts may be used according to the invention both in pure and in dilute form. Where used in diluted form, they conventionally contain approx. 2 to 80 wt. % active substance and, as solvent, the extracting agent or extracting agent mixture used to isolate them.

It may be preferred to use mixtures of two or more, particularly two, different plant extracts in agents according to the invention.

Ectoin, or ectoin derivatives, allantoin, taurine and/or bisabolol are furthermore suitable as s conditioner.

According to the invention, “ectoin and ectoin derivatives” refer to compounds of the formula (IV)—

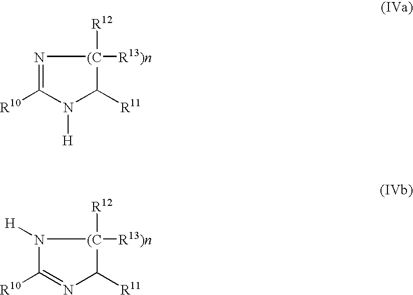

and/or the physiologically acceptable salts thereof and/or an isomeric or stereoisomeric form, wherein R¹⁰ is hydrogen, a branched or unbranched C₁-C₄ alkyl residue or a C₂-C₄ hydroxyalkyl residue; R¹¹ is hydrogen, a —COOR¹⁴ grouping or a —CO(NH)R¹⁴ grouping, wherein R¹⁴ may be hydrogen, a C₁-C₄ alkyl residue, an amino acid residue, a dipeptide or tripeptide residue; R¹² and R¹³ mutually independently are hydrogen, a C₁-C₄ alkyl residue or a hydroxyl group, with the proviso that both residues must not simultaneously be a hydroxyl group; and n is an integer from 1 to 3.

Suitable physiologically acceptable salts of the general compounds of formula (IVa) or (IVb) include alkali metal, alkaline earth metal, ammonium, triethylamine or tris-(2-hydroxyethyl)amine salts and those obtained from the reaction of compounds of formula (IVa) or (IVb) with inorganic and organic acids such as hydrochloric acid, phosphoric acid, sulfuric acid, branched or unbranched, substituted or unsubstituted (for example, by one or more hydroxyl groups) C₁-C₄ mono- or dicarboxylic acids, aromatic carboxylic acids and sulfonic acids such as acetic acid, citric acid, benzoic acid, maleic acid, fumaric acid, tartaric acid and p-toluenesulfonic acid. Examples of particularly preferred physiologically acceptable salts are the Na, K, Mg and Ca and ammonium salts of the compounds of the formula (IVa) or (IVb), and the salts which arise by reacting compounds of the formula (IVa) or (IVb) with hydrochloric acid, acetic acid, citric acid and benzoic acid.

Isomeric or stereoisomeric forms of the compounds according to the formula (IVa) or (IVb) are taken according to the invention to mean all occurring optical isomers, diastereomers, racemates, zwitterions, cations or mixtures thereof.

The term amino acid refers to the stereoisomeric forms, for example D- and L-forms, of the following compounds: asparagine, arginine, aspartic acid, glutamine, glutamic acid, β-alanine, γ-aminobutyrate, N_{ε}acetyllysine, N_{δ}-acetylornithine, N_{γ}-acetyldiaminobutyrate, N_{α}-acetyldiaminobutyrate, histidine, isoleucine, leucine, methionine, phenylalanine, serine, threonine and tyrosine. L-amino acids are preferred. Amino acid residues are derived from the corresponding amino acids. The following amino acid residues are preferred: Gly, Ala, Ser, Thr, Val, β-Ala, γ-aminobutyrate, Asp, Glu, Asn, Aln, N_{ε}acetyllysine, N_{δ}-acetylornithine, N_{γ}-acetyldiaminobutyrate, N_{α}-acetyldiaminobutyrate.

The amino acids have been abbreviated in line with the usual spelling. Chemically, the di- or tripeptide residues are acid amides and, on hydrolysis, break down into 2 or 3 amino acids. The amino acids in the di- or tripeptide residue are joined to one another by amide bonds.

Examples of C₁-C₄ alkyl groups in the compounds of the formula (IV) are methyl, ethyl, n-propyl, isopropyl, n-butyl, isobutyl and tert.-butyl. Preferred alkyl groups are methyl and ethyl; methyl is a particularly preferred alkyl group. Preferred C₂-C₄ hydroxyalkyl groups are the groups 2-hydroxyethyl, 3-hydroxypropyl or 4-hydroxybutyl; 2-hydroxyethyl is a particularly preferred hydroxyalkyl group.

The agents according to the invention preferably contain these conditioners in quantities of 0.001 to 2, in particular of 0.01 to 0.5 wt. %, in each case relative to the total ready-to-use preparation.

Mono- or oligosaccharides may also be used as a conditioner in the agents according to the invention.

Both monosaccharides and oligosaccharides such as cane sugar, lactose and raffinose may be used. Use of monosaccharides is preferred according to the invention. Among the monosaccharides, those compounds containing 5 or 6 carbon atoms are preferred.

Suitable pentoses and hexoses include ribose, arabinose, xylose, lyxose, allose, altrose, glucose, mannose, gulose, idose, galactose, talose, fucose and fructose. Arabinose, glucose, galactose and fructose are preferred carbohydrates. Glucose, which is suitable either in D-(+)- or L-(−)-configuration or as a racemate, is particularly preferred.

Derivatives of these pentoses and hexoses, such as the corresponding -onic and -uronic acids (saccharic acids), sugar alcohols and glycosides, may furthermore be used according to the invention. Preferred saccharic acids include gluconic acid, glucuronic acid, saccharic acid, mannosaccharic acid and mucic acid. Preferred sugar alcohols include sorbitol, mannitol and dulcitol. Preferred glycosides include the methyl glucosides.

Since the mono- or oligosaccharides used are conventionally obtained from natural raw materials such as starch, they generally exhibit the configurations corresponding to these raw materials (e.g., D-glucose, D-fructose and D-galactose).

The mono- or oligosaccharides are preferably present in hair treatment agents according to the invention in an amount of from 0.1 to 8.0 wt. %, preferably from 1.0 to 5.0 wt. %, based on total weight of the ready-to-use preparation.

The agent may furthermore contain at least one lipid as conditioner.

Lipids which are suitable according to the invention are phospholipids, for example, soy lecithin, egg lecithin and cephalins, as well as the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are distributed, for example, by Mona under the trade names Phospholipid EFA®, Phospholipid PTC® and Phospholipid SV®.

Agents according to the invention preferably contain lipids in an amount of from 0.01 to 10.00 wt. %, in particular, from 0.1. to 5.00 wt. %, based on total weight of the ready-to-use preparation.

Oil bodies are furthermore suitable as a conditioner.

Natural and synthetic cosmetic oil bodies include, for example—
vegetable oils. Examples of such oils include sunflower oil, olive oil, soy oil, rapeseed oil, almond oil, jojoba oil, orange oil, wheat germ oil, peach stone oil and the liquid fractions of coconut oil. However, other triglyceride oils such as the liquid fractions of beef fat together with synthetic triglyceride oils are also suitable.
liquid paraffin oils, isoparaffin oils and synthetic hydrocarbons and di-n-alkyl ethers having a total of from 12 to 36 C atoms, in particular 12 to 24 C atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, di-iso-pentyl ether, di-3-ethyldecyl ether, tert.-butyl-n-octyl ether, iso-pentyl-noctyl ether and 2-methylpentyl-n-octyl ether. The compounds 1,3-di-(2-ethylhexyl)cyclohexane (Cetiol® S) and di-n-octyl ether (Cetiol® OE) available as commercial products may be preferred.
ester oils. Ester oils refer to the esters of C₆-C₃₀ fatty acids with C₂-C₃₀ fatty alcohols. The monoesters of fatty acids with alcohols having 2 to 24 C atoms are preferred. Examples of fatty acid moieties used in the esters include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof which are obtained, for example, on pressure splitting of natural fats and oils, on oxidation of aldehydes from Roelen's oxo synthesis or on dimerization of unsaturated fatty acids. Examples of fatty alcohol moieties in the ester oils include isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, myristyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which are obtained, for example, on high pressure hydrogenation of technical methyl esters based on fats and oils or aldehydes from Roelen's oxo synthesis and as the monomer fraction on dimerization of unsaturated fatty alcohols. Particular preference is given according to the invention to isopropyl myristate IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate.
symmetrical, asymmetrical or cyclic esters of carbonic acid with fatty alcohols, for example described in DE-OS197 56 454, glycerol carbonate or dicaprylyl carbonate (Cetiol® CC).
trifatty acid esters of saturated and/or unsaturated linear and/or branched fatty acids with glycerol.
fatty acid partial glycerides, which refer to monoglycerides, diglycerides and the technical mixtures thereof. When using technical products, small quantities of triglycerides may still be contained therein depending on the production method. Partial glycerides preferably comply with the formula (D4-I)—

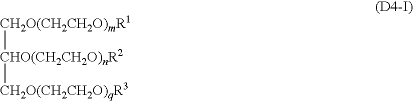

wherein R¹, R² and R³ mutually independently are hydrogen or a linear or branched, saturated and/or unsaturated acyl residue with 6 to 22, preferably 12 to 18, carbon atoms, with the proviso that at least one of these groups is an acyl residue and at least one of these groups is hydrogen. The sum (m+n+q) is 0 or numbers from 1 to 100, preferably 0 or 5 to 25. Preferably, R¹ is an acyl residue, R² and R³ are hydrogen, and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof. Preferably, oleic acid monoglycerides are used.

The input quantity of the natural and synthetic cosmetic oil bodies in the agents according to the invention conventionally amounts to 0.1-30 wt. % relative to the total ready-to-use preparation, preferably 0.1-20 wt. %, and in particular 0.1-15 wt. %.

The agents may moreover contain an enzyme as conditioner. Enzymes which are particularly preferred according to the invention are selected from a group which is formed of proteases, lipases, transglutaminase, oxidases and peroxidases.

Pearl extracts are also suitable as a conditioner.

Mussel pearls substantially consist of inorganic and organic calcium salts, trace elements and proteins. Pearls can be obtained from cultured mussels, which can be cultured in both fresh water and seawater. This may have an impact on the constituents of the pearls. A pearl extract preferred according to the invention is one which originates from mussels cultured in seawater or salt water. The pearls consist to a great extent of aragonite (calcium carbonate), conchiolin and an albuminoid. The latter components are proteins. Pearls furthermore additionally contain magnesium and sodium salts, inorganic silicon compounds and phosphates.

Pearl extract is produced by pulverizing the pearls. The pulverized pearls are then extracted using conventional methods. Extracting agents for producing the pearl extracts may comprise water, alcohols and mixtures thereof. Water refers to both demineralized water and seawater. Preferred alcohols include lower alcohols such as ethanol and isopropanol, but in particular polyhydric alcohols such as glycerol, diglycerol, triglycerol, polyglycerol, ethylene glycol, propylene glycol and butylene glycol, both as sole extracting agent and in a mixture with demineralized water or seawater. Pearl extracts based on water/glycerol mixtures have proved particularly suitable. Depending on the extraction conditions, the pearl proteins (conchiolin and albuminoid) may be present mostly in their native state, or already partially or mostly as protein hydrolyzates. A preferred pearl extract is one in which conchiolin and albuminoid already assume partially hydrolyzed form. The amino acids of these proteins include glutamic acid, serine, alanine, glycine, aspartic acid and phenylalanine. In a further particularly preferred development, it may be advantageous for the pearl extract to also be enriched with at least one or more of these amino acids. In a most preferred embodiment, the pearl extract is enriched with glutamic acid, serine and leucine. Furthermore, depending on the extraction conditions, in particular, depending on the extracting agent selected, the extract can contain a greater or lesser proportion of minerals and trace elements. One preferred extract contains organic and/or inorganic calcium salts and magnesium and sodium salts, inorganic silicon compounds and/or phosphates. One very particularly preferred pearl extract contains at least 75%, preferably 85%, particularly preferably 90% and very particularly preferably 95% of all the constituents of the naturally occurring pearls. Examples of pearl extracts usable according to the invention include the commercial products Pearl Protein Extract BG® (Lemanchem) or Crodarom® Pearl (Croda).

The above-described pearl extracts are preferably present in a quantity of from at least 0.01 up to 20 wt. %. The quantities used of the extract are preferably 0.01 up to 10 wt %, more preferably 0.01 to 5 wt. % based on total weight of the two-component agent.

Although each of the stated conditioners alone gives rise to a satisfactory result, the present invention also includes any embodiments in which the agent contains a plurality of conditioners, including from different groups.

The agent according to the invention can be present in a cosmetically acceptable carrier. This preferably comprises an aqueous, an alcoholic or an aqueous/alcoholic medium preferably containing at least 10 wt. % of water relative to the total preparation. Alcohols which may be present are, in particular, lower alcohols with 1 to 4 carbon atoms such as ethanol and isopropanol, conventionally used for cosmetic purposes.

Additional cosolvents which may be present include organic solvents or a mixture of solvents with a boiling point of below 400° C. in a quantity of 0.1 to 15 weight percent, preferably 1 to 10 weight percent based on total weight of the preparation. Particularly suitable additional cosolvents include unbranched or branched hydrocarbons such as pentane, hexane and isopentane, and cyclic hydrocarbons such as cyclopentane and cyclohexane. Further, particularly preferred water-soluble solvents include glycerol, ethylene glycol and propylene glycol in a quantity of up to 30 weight percent based on total weight of the preparation.

Agents according to the invention may be formulated in any forms conventional for cosmetic agents, for example, in the form of solutions, which can be applied onto the hair in a variety of forms such as a hair lotion or pump or aerosol spray, creams, emulsions, waxes, gels or surfactant-containing foaming solutions or other preparations suitable for application onto the skin or hair. The preparations preferably exhibit a pH value of 2 to 11. The pH range from 2 to 8 is particularly preferred. Details regarding pH value here relate for the purposes of the present document to the pH value at 25° C. unless otherwise stated.

Agents according to the invention preferably comprise hair treatment agents.

Hair creams and hair gels generally contain structuring agents and/or thickening polymers which serve to impart the desired consistency to the products. Structuring agents and/or thickening polymers are typically used in a quantity of 0.1 to 10 wt. %, relative to total weight of the ready-to-use preparation. Quantities of 0.5 to 5 wt. %, in particular of 0.5 to 3 wt. %, are very particularly preferred.

Corresponding hair treatment agents may also be formulated as a pump spray, aerosol spray, pump hair mousse or aerosol hair mousse.

Hair mousses refer to compositions which form a mousse on discharge from a suitable container. It may be necessary to add ingredients which promote mousse formation or which stabilize a mousse once it has formed. Surfactants and/or emulsifiers are in particular suitable for this purpose.

Where the products according to the invention comprise an aerosol product, these necessarily contain a propellant.

Propellants which are suitable according to the invention include N₂O, dimethyl ether, CO₂, air and alkanes with 3 to 5 carbon atoms, such as propane, n-butane, iso-butane, n-pentane, and iso-pentane, and mixtures thereof. Dimethyl ether, propane, n-butane, iso-butane and mixtures thereof are preferred.

According to a preferred embodiment, alkanes, mixtures of alkanes or mixtures of the stated alkanes with dimethyl ether are used as the sole propellant. The invention does, however, also include co-use of propellants of the chlorofluorocarbon type, especially fluorocarbon propellants.

With a given spray device, the size of the aerosol particles or of the mousse bubbles and the respective size distribution may be established by the quantity ratio of propellant to other components of the preparations.

Amount of propellant used varies as a function of the specific composition of the agent, of the packaging used and of the desired product type, for instance, hair spray or hair mousse. When using conventional spray devices, aerosol mousse products preferably contain the propellant in quantities of 1 to 35 wt. %, relative to total weight of the product. Quantities of 2 to 30 wt. %, in particular 3 to 15 wt. %, are particularly preferred. Aerosol sprays generally contain larger quantities of propellant. In this case, the propellant is preferably used in an amount of from 30 to 98 wt. %, relative to total weight of the product. Quantities of 40 to 95 wt. %, in particular 50 to 95 wt. %, are particularly preferred.

Aerosol products may be manufactured in conventional manner. In general, all components of the particular agent, with the exception of propellant, are introduced in a suitable pressure-resistant container. The container is then closed with a valve. The desired quantity of propellant is then introduced using conventional methods.

With addition of a UV filter, both the agents themselves and the treated body parts (here the keratin fibers) may be protected from the harmful effects of UV radiation. Therefore, at least one UV filter is preferably added to the agent. Suitable UV filters are not subject to any general restrictions with regard to structure and physical properties. Rather, any UV filter usable in the field of cosmetics whose absorption maximum is in the UVA (315-400 nm), the UVB (280-315 nm) and/or the UVC (<280 nm) range is suitable. UV filters with an absorption maximum in the UVB range, in particular in the range from approx. 280 to approx. 300 nm, are particularly preferred.

UV filters preferred according to the invention include substituted benzophenones, p-aminobenzoic acid esters, diphenylacrylic acid esters, cinnamic acid esters, salicylic acid esters, benzimidazoles and o-aminobenzoic acid esters.

Examples of suitable UV filters according to the invention include 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and the potassium, sodium and triethanolamine salts thereof (Phenylbenzimidazole sulfonic acid; Parsol® HS; Neo Heliopan® Hydro), 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)propane-1,3-dione (Butyl methoxydibenzoylmethane; Parsol® 1789, Eusolex® 9020), α-(2-oxoborn-3-ylidene)-toluene-4-sulfonic acid and the salts thereof, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Salicylat; Escalol® 587, Neo Heliopan® OS, Uvinul® 018), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone 5-sulfonic acid and the sodium salt thereof (Benzophenone-4; Uvinul® MS 40; Uvasorb® S 5), 3-(4′-methylbenzylidene)-D,L-camphor (4-Methylbenzylidene camphor; Parsol® 5000, Eusolex® 6300), 3-benzylidenecamphor (3-benzylidene camphor), 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)[2-oxoborn-3-ylidenemethyl]benzyl}-acrylamide, 2,4-dihydroxybenzophenone (Benzophenone-1; Uvasorb® 20H, Uvinul® 400), 1,1′-diphenylacrylonitrile acid 2-ethylhexyl ester (Octocrylene; Eusolex® OCR®, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid 2′-ethylhexyl ester. Preference is given to 4-aminobenzoic acid, N,N,N-trimethyl-4-(2-oxoborn-3-ylidenemethyl)aniline methyl sulfate, 3,3,5-trimethyl cyclohexyl salicylate, 2-hydroxy-4-methoxybenzophenone, 2-phenylbenzimidazole-5-sulfonic acid and the potassium, sodium and triethanolamine salts thereof, 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)propane-1,3-dione, α-(2-oxoborn-3-ylidene)-toluene-4-sulfonic acid and the salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone 5-sulfonic acid and the sodium salt thereof, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)[2-oxoborn-3-ylidenemethyl]benzyl}-acrylamide. Very particular preference is given according to the invention to 2-hydroxy-4-methoxybenzophenone, 2-phenylbenzimidazole-5-sulfonic acid and the potassium, sodium and triethanolamine salts thereof, 1-(4-tert.-butylphenyl)-3-(4-methoxyphenyl)-propane-1,3-dione, 4-methoxycinnamic acid 2-ethylhexyl ester and 3-(4′-methylbenzylidene)-D,L-camphor.

UV filters whose molar extinction coefficient at the absorption maximum is above 15,000, in particular above 20,000, are preferred.

It has additionally been found that, in the case of structurally similar UV filters, often the water-insoluble compound performs better versus those water-soluble compounds which differ therefrom by one or more additionally ionic groups. UV filters which are considered to be water-insoluble include those which at 20° C. are only 1 wt. %, in particular no more than 0.1 wt. %, soluble in water. Furthermore, these compounds should be at least 0.1, in particular at least 1 wt. %, soluble in conventional cosmetic oil components at room temperature. Use of water-insoluble UV filters may therefore be preferred according to the invention.

According to a further embodiment of the invention, preferred UV-filters include those having a cationic group, in particular a quaternary ammonium group.

These UV filters have the general structure U-Q.

The structural element U therein is a UV radiation-absorbing group. This group may be derived in principle from known, previously indicated UV filters usable in the field of cosmetics, in which a group (generally a hydrogen atom) of the UV filter is replaced by a cationic group Q, in particular with a quaternary amino function.

Compounds from which the structural element U may be derived include—

substituted benzophenones,

p-aminobenzoic acid esters,

diphenylacrylic acid esters,

cinnamic acid esters,

salicylic acid esters,

benzimidazoles and

o-aminobenzoic acid esters.

Structural elements U which are derived from cinnamic acid amide or from N,N-dimethylaminobenzoic acid amide are preferred according to the invention.

The structural elements U may be selected so that the absorption maximum of the UV filters lies both in the UVA (315-400 nm) and in the UVB (280-315 nm) or in the UVC (<280 nm) range. UV filters with an absorption maximum in the UVB range, in particular from approx. 280 to approx. 300 nm, are particularly preferred.

Furthermore, the structural element U, also as a function of structural element Q, is preferably selected so that the molar extinction coefficient of the UV filter at the absorption maximum is above 15,000, in particular above 20,000.

The structural element Q preferably contains a quaternary ammonium group as the cationic group. This quaternary ammonium group may be linked directly to the structural element U so that the structural element U is one of the four substituents of the positively charged nitrogen atom. However, one of the four substituents on the nitrogen atom is preferably a group, in particular an alkylene group having 2 to 6 carbon atoms, which functions as a link between the structural element U and the positively charged nitrogen atom.

Advantageously, the group Q has the general structure —(CH₂)ₓ—N⁺R¹R²R³X⁻, wherein x is an integer from 1 to 4; R¹ and R² mutually independently are C₁₋₄ alkyl groups; R³ is a C₁₋₂₂ alkyl group or a benzyl group; and X⁻ is a physiologically acceptable anion. In the context of this general structure, x is preferably the number 3, R¹ and R² in each case is a methyl group, and R³ is either a methyl group or a saturated or unsaturated, linear or branched, hydrocarbon chain having 8 to 22, in particular 10 to 18, carbon atoms.

Physiologically acceptable anions include inorganic anions such as halides, particularly chloride, bromide and fluoride, sulfate ions and phosphate ions and organic anions such as lactate, citrate, acetate, tartrate, methosulfate and tosylate.

Two preferred UV filters with cationic groups are the commercially obtainable compounds cinnamic acid amidopropyltrimethylammonium chloride (Incroquat® UV-283) and dodecyldimethylaminobenzamidopropyldimethylanunonium tosylate (Escalol® HP 610).

Agents according to the invention can also include a combination of two or more UV filters. In this embodiment, combination of at least one water-insoluble UV filter with at least one UV filter having a cationic group is preferred.

UV filters are conventionally present in quantities of from 0.01 to 5.00 wt. %, based on total weight of the ready-to-use preparation. Quantities of from 0.1 to 2.5 wt. % are preferred.

For the preferred case in which the agent according to the invention is a hair treatment agent, the hair treatment agent may contain in one embodiment one or more direct dyes. This makes it possible for the treated keratin fibers not only to be temporarily structured but also to be dyed at the same time when applying the composition. This may be particularly desirable when dyeing only temporarily, for example, when conspicuous fashion colors are desired, which may be removed from the keratin fiber simply by washing.

Common direct dyes include nitrophenylenediamines, nitroaminophenols, azo dyes, anthraquinones or indophenols. Preferred direct dyes include the compounds known by the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black Land Acid Black 52 as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-1-hydroxy-4-nitrobenzene.

Cationic direct dyes are preferably used. Particular preference is here given to
(a) cationic triphenylmethane dyes, such as Basic Blue 7, Basic Blue 26, Basic Violet 2 and Basic Violet 14;
(b) aromatic systems substituted with a quaternary nitrogen group, such as Basic Yellow 57, Basic Red 76, Basic Blue 99, Basic Brown 16 and Basic Brown 17; and
(c) direct dyes containing at least one heterocycle having at least one quaternary nitrogen atom, such as are mentioned in claims 6 to 11 of EP 0 998 908 A2.

Preferred cationic direct dyes of group (c) include the following compounds—

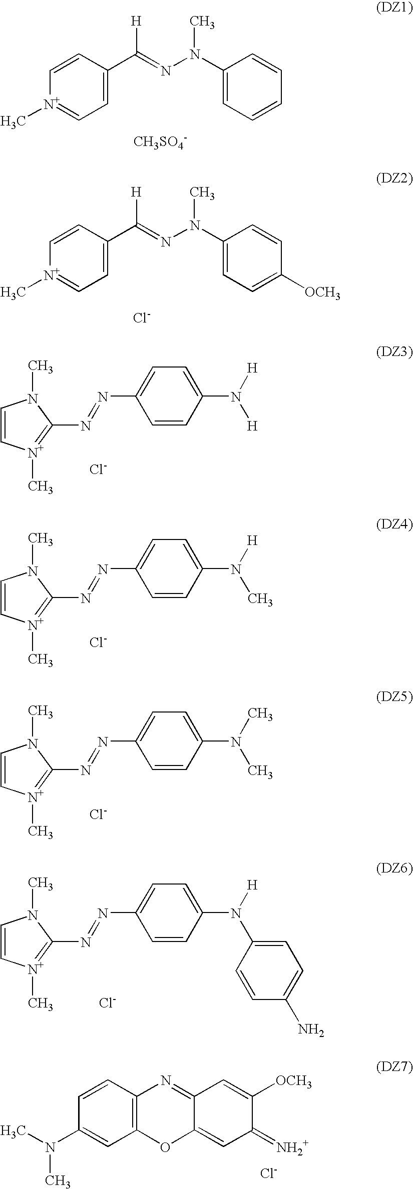

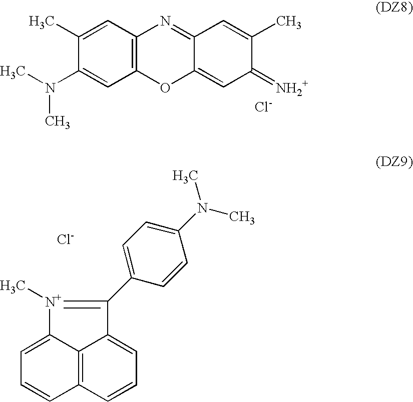

Compounds according to the formulae (DZ1), (DZ3) and (DZ5), also known by the names Basic Yellow 87, Basic Orange 31 and Basic Red 51, are very particularly preferred cationic direct dyes of group (c).

Cationic direct dyes distributed under the trademark Arianor® are cationic direct dyes which are likewise very particularly preferred according to the invention.

Agents according to this embodiment preferably contain direct dyes in a quantity of from 0.001 to 20 wt. %, based on total weight of the agent.

Agents according to the invention may also contain naturally occurring dyes found, for example, in henna red, henna neutral, henna black, chamomile flowers, sandalwood, black tea, alder buckthorn bark, sage, logwood, madder root, catechu, lotus tree and alkanet root.

It is not necessary for the direct dyes in each case to be uniform compounds. Instead, as a result of the production processes for the individual dyes, the agents according to the invention may contain subordinate quantities of still further components, provided that these do not have a disadvantageous effect on the styling result or must be excluded for other, for example toxicological, reasons.

In addition to the above mentioned components, agents according to the invention may also contain any active ingredients, additives and auxiliary substances known for such preparations.

In many cases, the agents contain at least one surfactant. Not only anionic, but also zwitterionic, ampholytic, nonionic and cationic surfactants are suitable. Often, however, it has proven advantageous to select the surfactants from among anionic, zwitterionic or nonionic surfactants.

Further active ingredients and auxiliary substances and additives include—
thickeners such as agar-agar, guar gum, alginates, xanthan gum, gum arabic, karaya gum, locust bean flour, linseed gums, dextrans, cellulose derivatives (e.g., methylcellulose, hydroxyalkylcellulose and carboxymethylcellulose), starch fractions and derivatives such as amylose, amylopectin and dextrins, clays such as bentonites, or completely synthetic hydrocolloids such as for example polyvinyl alcohol,
structuring agents such as maleic acid and lactic acid, perfume oils, dimethyl isosorbide and cyclodextrins,
solvents and solubilizing agents such as ethanol, isopropanol, ethylene glycol, propylene glycol, glycerol and diethylene glycol,
quaternized amines such as methyl-1-alkylamidoethyl-2-alkylimidazolinium methosulfate
defoamers such as silicones,
dyes for coloring the agent,
antidandruff active ingredients such as piroctone olamine, zinc omadine and climbazole,
substances for adjusting pH value, such as for example conventional acids, in particular edible acids and bases,
cholesterol,
consistency providers, such as sugar esters, polyol esters or polyol alkyl ethers,
fats and waxes such as spermaceti, beeswax, montan wax and paraffins,
fatty acid alkanolamides,
complexing agents such as EDTA, NTA, β-alaninediacetic acid and phosphonic acids,
swelling and penetrating substances such as glycerol, propylene glycol monoethyl ether, carbonates, hydrogencarbonates, guanidines, ureas and primary, secondary and tertiary phosphates,
opacifiers such as latex, styrene/PVP and styrene/acrylamide copolymers
pearlescent agents such as ethylene glycol mono- and distearate as well as PEG-3 distearate,
preservatives,
stabilizers for hydrogen peroxide and other oxidizing agents,
propellants such as propane/butane mixtures, N₂O, dimethyl ether, CO₂ and air,
antioxidants.

Concerning other optional components and the quantities of these components used, reference is made to relevant handbooks known to a person skilled in the art, for example, Kh. Schrader, Grundlagen and Rezepturen der Kosmetika (Fundamentals and Formulations of Cosmetics), 2^{nd} Ed., Hüthig Buch Verlag, Heidelberg, Germany (1989).

The present invention also provides for the use of agents according to the invention for treating keratin fibers, wherein this treatment preferably comprises temporary deformation.

Since agents according to the invention necessarily contain a film-forming and/or setting polymer, this type of use is particularly advantageous. The film-forming and/or setting polymers here not only regulate the effect of the pheromone used, but also build up and/or stabilize a desired hairstyle.

The following Examples are intended to further explain the subject matter of the present invention without limiting it in any way.

### EXAMPLES

The following formulations were produced. Quantities are in weight percent unless stated otherwise.

### 1—Styling Gel

Styling gels (Invention 1 to Invention 3) according to the invention were produced in accordance with the following table—

| Raw materials | Invention 1 | Invention 2 | Invention 3 |
|---|---|---|---|
| 4,16-Androstadien-3-one | 0.0003 | — | 0.0002 |
| 1,3,5(10),16-Estratetraen-3-ol | — | 0.0003 | 0.0002 |
| Fixate ™ G-100¹ | 15.00 | 15.00 | 15.00 |
| Synthalen K² | 1.00 | 1.00 | 1.00 |
| Dekafald³ | 0.10 | 0.10 | 0.10 |
| Triethanolamine | 0.70 | 0.70 | 0.70 |
| PEG-40 Hydrogenated | 0.41 | 0.41 | 0.41 |
| Castor Oil⁴ | | | |
| Perfume | 0.15 | 0.15 | 0.15 |
| Water, deionized | ad 100 | ad 100 | ad 100 |

¹Aminomethylpropanol salt of a copolymer of allyl methacrylate with one or more monomers selected from acrylic acid, methacrylic acid, acrylic acid esters and methacrylic acid esters (aqueous solution with a polymer content of 25-28 wt. %; INCI name: Aqua, AMP-Acrylates/Allyl Methacrylate Copolymer) (Noveon)
²Acrylic acid homopolymer linked by means of pentaerythritol, sucrose or propylene allyl ether (INCI name: Carbomer) (3V Sigma)
³1,3-Dihydroxymethyl-5,5-dimethylhydantoin (approx. 54-56 wt. % active substance in water; INCI name: DMDM Hydantoin) (Jan Dekker)
⁴Polyethylene glycol derivative of hydrogenated castor oil with on average 40 mol of ethylene oxide (INCI name: PEG-40 Hydrogenated Castor Oil) (BASF)

### 2—Styling Mousse

The formulation according to the invention for a styling mousse (Invention 4) was produced according to the following table—

| Raw materials | Invention 4 |
|---|---|
| 4,16-Androstadien-3-one | 0.0004 |
| Fixate ™ G-100¹ | 15.00 |
| Brij ® 30⁵ | 0.50 |
| Dekafald³ | 0.10 |
| PEG-40 Hydrogenated Castor Oil⁴ | 0.41 |
| Perfume | 0.15 |
| Water, deionized | ad 100 |

⁵Polyethylene glycol monolauryl ether, dodecanol with approx. 4 mol equivalent of ethylene oxide (INCI name: Laureth-4) (Uniquema)

Although the present invention has been described in detail, it is to be clearly understood that the same is by way of illustration and example only, and is not to be taken as a limitation. The spirit and scope of the present invention are to be limited only by the terms of any claims presented hereafter.

We claim:

1. Cosmetic agent comprising, in a cosmetically acceptable carrier:
(a) 1×10⁻⁷ to 1×10⁻² wt. % of at least one steroid chosen from 16-androstene steroids according to formula (I)— 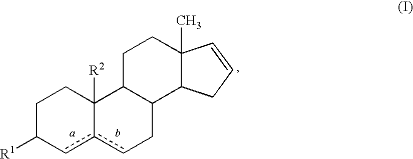
wherein R¹ is an oxo group, a hydroxyl group, a C₁-C₆ alkoxy, an acyloxy, or a benzoyloxy group; R² is hydrogen, a hydroxyl group, a C₁-C₆ alkyl, hydroxy-C₁-C₆-alkyl, C₁-C₆-alkoxy-C₁-C₆-alkyl, C₁-C₆ alkoxy, acyl, acyloxy, acyl-C₁-C₆-alkyl or acyloxy-C₁-C₆-alkyl group; and the dashed lines a and b represent alternative positions of a double bond; and
estrene steroids according to formula (II)— 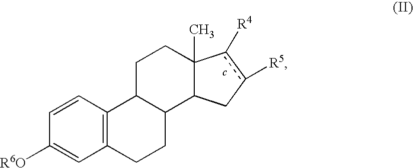
wherein R⁴ is an oxo group, hydrogen, a hydroxyl group, a C₁-C₆ alkyl group, sulfate, a 3-cyclopentylpropionate group or acetate; R⁵ is hydrogen or a hydroxyl group; R⁶ is hydrogen, a C₁-C₆ alkyl group, a benzoyl, 3-cyclopentylpropionate, acetyl, C₁-C₆ acyl or a sulfate group; and the dashed line c represents an optional double bond; and
(b) 0.1 to 30.0 wt. % of at least one film-forming and/or setting polymer.

2. Cosmetic agent according to claim 1, wherein the at least one steroid is at least a 16-androstene steroid according to formula (I) and wherein R¹ is an oxo group, a hydroxyl group, a methoxy, an acetoxy, a propionoxy or a benzoyloxy group.

3. Cosmetic agent according to claim 1, wherein the at least one steroid is at least a 16-androstene steroid according to formula (I) and wherein R² is hydrogen, a hydroxyl group, a methyl, ethyl, n-propyl, iso-propyl, methoxy or ethoxy group.

4. Cosmetic agent according to claim 1, wherein the at least one steroid is at least a 16-androstene steroid and the 16-androstene steroid is 4,16-androstadien-3-one.

5. Cosmetic agent according to claim 1, wherein the at least one steroid is at least an estrene steroid according to formula (II) and wherein R⁴ is an oxo group, a hydroxyl group or hydrogen.

6. Cosmetic agent according to claim 1, wherein the at least one steroid is at least an estrene steroid according to formula (II) and wherein R⁵ is hydrogen or a hydroxyl group.

7. Cosmetic agent according to claim 1, wherein the at least one steroid is at least an estrene steroid according to formula (II) and wherein R⁶ is hydrogen.

8. Cosmetic agent according to claim 1, wherein the at least one steroid is at least an estrene steroid according to formula (II) and the estrene steroid is 1,3,5(10)-estratriene-3,17β-diol, 1,3,5(10)-estratiene-3,16α,17β-triol, 1,3,5(10)-estratrien-3-ol-17-one and/or 1,3,5(10),16-estratetraen-3-ol.

9. Cosmetic agent according to claim 1 wherein the at least one steroid is a steroid mixture of at least one 16-androstene steroid according to formula (I) and at least one estrene steroid according to formula (II).

10. Cosmetic agent according to claim agent according to claim 1, wherein the at least one steroid is present in an amount of from 1×10⁻⁴ to 5×10⁻³ wt. %.

11. Cosmetic agent according to claim agent according to claim 10, wherein the at least one steroid is present in an amount of from 3×10⁻⁴ to 1×10⁻³ wt. %.

12. Cosmetic agent according to claim 1, wherein the at least one film-forming and/or setting polymer is present in an amount of from 0.5 to 25.0 wt. %.

13. Cosmetic agent according to claim 1, wherein the at least one film-forming and/or setting polymer is chosen from aminomethylpropanol salts of copolymers of allyl methacrylate with one or more monomers chosen from acrylic acid, methacrylic acid, acrylic acid esters and methacrylic acid esters; vinylpyrrolidone-vinyl acetate copolymers; vinylpyrrolidone-vinylcaprolactam-dimethylaminopropylacrylamide copolymers; copolymers of octylacrylamide with t-butylaminoethyl methacrylate and two or more monomers selected from acrylic acid, methacrylic acid, acrylic acid esters and methacrylic acid esters; and copolymers of C₁₋₂ alkyl succinates with hydroxyalkyl acrylates and one or more monomers selected from acrylic acid, methacrylic acid, acrylic acid esters and methacrylic acid esters.

14. Cosmetic agent according to claim 13, wherein the at least one film-forming and/or setting polymer is at least an aminomethylpropanol salt of a copolymer of alkyl methacrylate with one or more monomers chosen from acrylic acid, methacrylic acid, acrylic acid esters and methacrylic acid esters.

15. Method of treating keratin fibers comprising applying the cosmetic agent of claim 1 to keratin fibers.
